(19) United States
(12) Patent Application Publication
(10) Pub. No.: US 2024/0161869 A1
(43) Pub. Date: May 16, 2024

(54) SYSTEMS AND METHODS FOR ALGORITHMICALLY ESTIMATING PROTEIN CONCENTRATIONS

(71) Applicant: Clara Foods Co., Daly City, CA (US)

(72) Inventors: A Samuel POTTINGER, Daly City, CA (US); Dane Mathias JACOBSON, Newton, MA (US); Ranjan PATNAIK, Daly City, CA (US); Zachary FRIAR, Redwood City, CA (US)

(73) Assignee: Clara Foods Co., Daly City, CA (US)

(21) Appl. No.: 18/513,505

(22) Filed: Nov. 17, 2023

Related U.S. Application Data
(63) Continuation of application No. PCT/US2022/030288, filed on May 20, 2022.
(60) Provisional application No. 63/191,264, filed on May 20, 2021.

(51) Int. Cl. G16B 25/10; G16B 30/10; G16B 40/20

(52) U.S. Cl. CPC G16B 25/10; G16B 30/10; G16B 40/20

(57) ABSTRACT

Disclosed is a computer-implemented method and system for estimating protein concentrations. The method comprises first generating a synthetic dataset based at least on protein signature or fingerprint data. Then, the method comprises training a model using in part the synthetic dataset, without requiring protein-specific calibration or training. Finally, the method comprises using the model to estimate or predict a percentage amount of a specific protein of interest (POI) in one or more heterogeneous samples, even if the POI was not used in modeling at the time of training.

### CROSS-REFERENCE

This application is a continuation of International Application No. PCT/US2022/030288, filed on May 20, 2022, which claims priority to U.S. Provisional Application No. 63/191,264, filed on May 20, 2021, the contents of each of which is incorporated by reference herein in its entirety.

### BACKGROUND

Advances in modern chemistry and biological sciences have made it possible to synthesize biological products comprising animal proteins for such wide-ranging uses as plant-based eggs, lab-grown meat, improved baking yeast, and nutritional supplements. To do so, researchers and clinicians need to know the protein compositions of the biological products they wish to synthesize. Current methods of determining these protein compositions may be costly and labor-intensive, requiring skilled personnel and lab equipment to perform techniques such as X-ray crystallography or spectrometry on protein samples. Machine learning analysis has been used to make predictions from analysis of biological samples, showing promise for mitigating the need to use expensive laboratory techniques.

### SUMMARY

There is a need to develop a cost-effective and less labor-intensive solution for determining the protein makeup of a biological sample. It is possible to determine the protein composition of a sample by determining the amino acid composition of a sample. This may be done using amino acid analysis (AAA). Traditional methods of performing AAA include hydrolyzing a biological sample to release the amino acids, and then performing a reaction to produce signals indicating which amino acids are present in the biological sample. The systems and methods disclosed herein can utilize machine learning analysis to make predictions from analysis of biological samples, thereby mitigating the need to use expensive laboratory techniques. For example, the systems and methods herein can predict a presence of a protein of interest (POI) and what percentage of protein is the POI (POI %) by training a machine learning algorithm using amino acid analysis (AAA) data. Synthesized AAA data can be used in the training, which produces robust prediction results when the model is tested as described in detail herein.

An aspect of the present disclosure is a computer-implemented method for estimating protein concentrations in one or more heterogeneous samples. The method comprises steps of generating a synthetic dataset based at least on protein signature or fingerprint data; training a model using in part the synthetic dataset, without requiring protein-specific calibration or training; and using the model to estimate or predict a percentage amount of a specific protein of interest (POI) in one or more heterogeneous samples.

In embodiments, the protein signature or fingerprint data comprises amino acid analysis (AAA) data.

In some embodiments, the protein signature or fingerprint data comprises high performance liquid chromatography (HPLC) or infrared spectroscopy (IR)-based data.

In various embodiments, the model is useable to predict or estimate a plurality of different POIs in a plurality of different heterogeneous samples.

In embodiments, the method further comprises using the model to predict one or more POIs that are not present in the synthetic dataset or that are not used in training the model.

In some embodiments, the POI % is estimated or predicted using the model in substantially less time, e.g., a day or less, and utilizing substantially less resources compared to high performance liquid chromatography (HPLC), which normally takes about a month to establish calibration for each per protein examined.

In various embodiments, the POI % is estimated or predicted by the model using amino acid, mass, or a mole percentage (%) of the specific POI.

In embodiments, the AAA data comprises amino acid, mass, or a mole percentage (%) distributions. In some cases, the amino acid mole % distributions are obtained from a set of FASTA files.

In some embodiments, the AAA data comprises theoretical AAA results at 100% purity.

In various embodiments, the synthetic dataset is generated through simulations by combining theoretical AAA values of different proteins expected at 100% purity, at different concentrations of the different proteins.

In embodiments, the synthetic dataset comprises weighted averages of theoretical AAA values of different proteins at 100% purity, wherein the weighted averages are generated by randomly applying a plurality of weights to the theoretical AAA values.

In some embodiments, the synthetic dataset comprises more than 1000 simulated theoretical AAA results.

In various embodiments, the training of the model is performed in one hour or less.

In embodiments, the model comprises a neural network. In some cases, the neural network is based in part on a pseudo-Siamese architecture. The pseudo-Siamese neural network architecture may comprise a pair of input vectors without corresponding parallel branches. The pair of input vectors may comprise (i) a first input vector comprising of historical or available AAA results and (ii) a second input vector comprising theoretical AAA results at 100% purity. In some cases, the historical or available AAA results are obtained from a first database of naturally occurring proteins inside hen egg white and a second database of common host cell proteins. In some embodiments, the host cell proteins are expressed by a microbe selected from a Pichia species, a Saccharomyces species, a Trichoderma species, a Pseudomonas species, an Aspergillus species, and an E. coli species; the Pichia species may be Pichia pastoris or the Saccharomyces species may be Saccharomyces cerevisiae.

In various embodiments, the neural network does not require learning of a lower dimensional representation.

In embodiments, a comparison function in the neural network is automatically learned without external human input or intervention.

In some embodiments, generating the synthetic dataset further comprises splitting the synthetic dataset into a training set, a validation set, and a test set. In some cases, training the model comprises using the training set in fitting the model, the test set is not provided to the model during the training of the model, or using the validation set to check a mean squared error (MAE) of the model and determining whether the MAE of the model meets a criteria threshold; the method may further comprise persisting the model to memory upon determining that the MAE of the model meets the criteria threshold.

In various embodiments, the model has a performance of a mean absolute error (MAE) of 3 points for a hidden test set of proteins within the synthetic dataset, and 6 points for novel proteins that are not present in the synthetic dataset.

In embodiments, the model is based on linear (lasso), support vector machine (SVM), decision tree, or random forest.

In some embodiments, the POI % is greater than or equal to about 50%.

In various embodiments, the POI % is less than about 50%.

In embodiments, the model is further trained using actual or real data collected over time. In some cases, the target product is a protein recombinantly expressed by a host cell. In some cases, the specific POI is a contaminant, e.g., a contaminant that is unintentionally included in the multi-protein sample and/or the specific POI is a process byproduct or an added protein.

In some embodiments, the specific POI is a target product. In some cases, the target product is a protein recombinantly expressed by a host cell. In some cases, the specific POI is a contaminant, e.g., a contaminant that is unintentionally included in the multi-protein sample and/or the specific POI is a process byproduct or an added protein.

Another aspect of the present disclosure is a system for estimating protein concentrations in one or more heterogeneous samples. The system comprises one or more processors; and a non-transitory computer readable medium storing a plurality of instructions, which when executed, cause the one or more processors to: (a) generate a synthetic dataset based at least on protein signature or fingerprint data; (b) perform training of a model using the synthetic dataset, without requiring protein-specific calibration or training; and (c) estimate or predict, using the model, a percentage amount of a specific protein of interest (POI) in the one or more heterogeneous samples.

In various embodiments, the specific POI is a target product.

In embodiments, the target product is a protein recombinantly expressed by a host cell.

In some embodiments, the specific POI is a contaminant. In some cases, the contaminant is unintentionally included in the multi-protein sample.

In various embodiments, the specific POI is a process byproduct or an added protein.

In embodiments, the multi-protein sample comprises a culturing medium for cultivating a host cell or the multi-protein sample is derived from a culturing medium used for cultivating a host cell. In some cases, the host cell is a microbial cell selected from a Pichia cell, a Saccharomyces cell, a Trichoderma cell, a Pseudomonas cell, an Aspergillus cell, and an E. coli cell; the Pichia cell may be a Pichia pastoris cell or the Saccharomyces cell may be a Saccharomyces cerevisiae cell.

Yet another aspect of the present disclosure is a computer-implemented method for estimating protein concentrations in one or more heterogeneous samples. The method comprises a step of using a model to estimate or predict a percentage amount of a specific protein of interest (POI) in one or more heterogeneous samples, wherein the model is obtained by: (a) generating a synthetic dataset based at least on protein signature or fingerprint data; (b) training the model using in part the synthetic dataset, without requiring protein-specific calibration or training.

In some embodiments, the protein signature or fingerprint data comprises amino acid analysis (AAA) data.

In various embodiments, the protein signature or fingerprint data comprises high performance liquid chromatography (HPLC) or infrared spectroscopy (IR)-based data.

In embodiments, the model is useable to predict or estimate a plurality of different POIs in a plurality of different heterogeneous samples.

In some embodiments, the method further comprises using the model to predict one or more POIs that are not present in the synthetic dataset or that are not used in training the model.

In various embodiments, the POI % is estimated or predicted using the model in substantially less time, e.g., a day or less, and utilizing substantially less resources compared to high performance liquid chromatography (HPLC), which normally takes about a month to establish calibration for each per protein examined.

In embodiments, the POI % is estimated or predicted by the model using amino acid, mass, or a mole percentage (%) of the specific POI.

In some embodiments, the AAA data comprises amino acid, mass, or a mole percentage (%) distributions. In some cases, the amino acid mole % distributions are obtained from a set of FASTA files.

In various embodiments, the AAA data comprises theoretical AAA results at 100% purity.

In embodiments, the synthetic dataset is generated through simulations by combining theoretical AAA values of different proteins expected at 100% purity, at different concentrations of the different proteins.

In some embodiments, the synthetic dataset comprises weighted averages of theoretical AAA values of different proteins at 100% purity, wherein the weighted averages are generated by randomly applying a plurality of weights to the theoretical AAA values.

In various embodiments, the synthetic dataset comprises more than 1000 simulated theoretical AAA results.

In embodiments, the training of the model is performed in one hour or less.

In some embodiments, the model comprises a neural network. In some cases, the neural network is based in part on a pseudo-Siamese architecture. The pseudo-Siamese neural network architecture may comprise a pair of concatenated input vectors without corresponding parallel branches. The pair of concatenated input vectors may comprise (i) a first input vector comprising of historical or available AAA results and (ii) a second input vector comprising theoretical AAA results at 100% purity. In various embodiments, the historical or available AAA results are obtained from a first database of naturally occurring proteins inside hen egg white and a second database of common host cell proteins. The host cell proteins may be expressed by a microbe selected from Pichia species, a Saccharomyces species, a Trichoderma species, a Pseudomonas species, an Aspergillus species, and an E. coli species; the Pichia species may be Pichia pastoris or the Saccharomyces species may be Saccharomyces cerevisiae.

In embodiments, the neural network does not require learning of a lower dimensional representation.

In some embodiments, a comparison function in the neural network is automatically learned without external human input or intervention.

In various embodiments, the step of generating the synthetic dataset further comprises splitting the synthetic dataset into a training set, a validation set, and a test set. In some embodiments, the training the model comprises using the training set in fitting the model, the test set is not provided to the model during the training of the model, the method further comprises using the validation set to check a mean squared error (MAE) of the model and determining whether the MAE of the model meets a criteria threshold, or the method further comprises persisting the model to memory upon determining that the MAE of the model meets the criteria threshold.

In embodiments, the model has a performance of a mean absolute error (MAE) of 3 points for a hidden test set of proteins within the synthetic dataset, and 6 points for novel proteins that are not present in the synthetic dataset.

In some embodiments, the model is based on linear (lasso), support vector machine (SVM), decision tree, or random forest.

In various embodiments, the POI % is greater than or equal to about 50%.

In embodiments, the POI % is less than about 50%.

In some embodiments, the model is further trained using actual or real data collected over time. In some cases, the target product is a protein recombinantly expressed by a host cell. The specific POI may be a contaminant, e.g., the contaminant is unintentionally included in the multi-protein sample.

In various embodiments, the specific POI is a target product. In some cases, the target product is a protein recombinantly expressed by a host cell. The specific POI may be a contaminant, e.g., the contaminant is unintentionally included in the multi-protein sample.

In embodiments, the specific POI is a process byproduct or an added protein.

In some embodiments, the model includes four layers of neurons, wherein the layers are of sizes 64, 32, 16, and 8 neurons.

In various embodiments, the model is trained using a ridge (L2) regularization of 0.1 and an Adam learning rate of 00001.

Any aspect or embodiment described herein can be combined with any other aspect or embodiment as disclosed herein.

Additional aspects and advantages of the present disclosure will become readily apparent to those skilled in this art from the following detailed description, wherein illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the disclosures are set forth with particularity in the appended claims. A better understanding of the features and advantages of the present disclosures will be obtained by reference to the following detailed description that sets forth illustrative embodiments, in which the principles of the present disclosures are utilized, and the accompanying drawings (also “Figure” and “FIG.” herein), of which:

FIG. 1 illustrates a system for determining a percentage of a protein of interest (POI %) in a biological sample in accordance with some embodiments;

FIG. 2 illustrates a pipeline for implementing a machine learning algorithm on a set of AAA data to determine a percentage of a protein of interest, in accordance with some embodiments;

FIG. 3 illustrates a process flow diagram 300 for predicting a POI % from AAA data, in accordance with some embodiments;

FIG. 4 illustrates an example neural network topology in accordance with some embodiments;

FIG. 5 illustrates an analysis of a likelihood that two proteins will have the same AAA signature, in accordance with some embodiments;

FIG. 6 illustrates the efficacy chart of the system at predicting a POI %, in accordance with some embodiments;

FIG. 7 illustrates results of an experiment examining model performance when the POI does not necessarily constitute a majority of the sample, in accordance with some embodiments;

FIG. 8 shows a computer system that is programmed or otherwise configured to implement methods provided herein;

FIG. 9 shows an evaluation for known protein performance at low cardinality;

FIG. 10 shows an evaluation of zero-shot learning at low cardinality; and

FIG. 11 illustrates two approaches for using AAA with a synthetic simulated dataset.

### DETAILED DESCRIPTION

While various embodiments of the present disclosures have been shown and described herein, it will be obvious to those skilled in the art that such embodiments are provided by way of example only. Numerous variations, changes, and substitutions may occur to those skilled in the art without departing from the scope of the present disclosures. It should be understood that various alternatives to the embodiments described herein may be employed.

Disclosed herein are systems and methods for algorithmically determining a percentage of a protein of interest (POI %) in a biological sample. The disclosed system can be used to predict a POI % using substantially less time and resources compared to existing experimental methods such as high performance liquid chromatography (HPLC). The biological sample may be a heterogeneous sample including one or more proteins and other biological components. The POI within the sample may be a target product, a contaminant unintentionally included in the sample, a process byproduct, or an added protein. A target product may be a protein recombinantly expressed by a cultured host cell, e.g., a plant cell, an animal cell, or a microbial cell (for example a fungal cell or bacterial cell). A process byproduct protein may be non-recombinantly expressed by the host cell components of the host cell itself. An added protein may be intentionally or inherently included in the sample. In some cases, a culturing medium inherently includes an added protein. In some cases, proteins are intentionally added to a culturing medium to promote growth of the host cells, e.g., proteins included in yeast extract, peptone, and natural or synthetic serum. In various embodiments, a protein may be intentionally added to a protein sample, as examples, to increase the total protein content of the sample, to increase the amount of specific POI in the sample, and to provide a stabilizing effect upon the POI.

The systems and methods herein can provide predictions by percent amino acids, percent mass, or percent moles. Percent amino acids calculations are based on how many amino acids are associated with a protein out of all amino acids in a sample; it uses the formula:

$${\%}_{aa} = \frac{{n}_{aapoi}}{{n}_{aatotal}}$$

This offers the benefit that the calculations do not need to consider amino acid weights and different sized proteins (in terms of amino acids). Percent by mass takes in account the different masses of different proteins; it uses the formula:
This approach helps in understanding yields, sensory, and functional properties. Calculations can

$${\%}_{mass} = \frac{{m}_{poi}}{{m}_{total}} = \frac{\sum {n}_{aapoi} * {m}_{aa}}{\sum {n}_{aatotal} * {m}_{aa}}$$

be based on percent of moles, according to the formula:

$${\%}_{mol} = \frac{{n}_{poi}}{\sum {n}_{protein}} = \frac{{n}_{poi}}{{n}_{total}}$$

Moreover, these purity calculations may be converted into other formats. For example, amino acid percentages can be converted into percent mass by the following formula:

$${\%}_{mass} = \frac{{\overline{m}}_{aapoi}}{{\overline{m}}_{aatotal}} * \frac{\sum {n}_{aapoi}}{\sum {n}_{aatotal}} = \frac{{\overline{m}}_{aapoi}}{{\overline{m}}_{aatotal}} * {\%}_{aa}$$

And, amino acid percentages can be converted into molar percent by the following formula:

In one embodiment, the system may determine the POI % from a protein signature comprising amino acid analysis (AAA) data. In other embodiments, the system may use a protein

$${\%}_{mol} = \frac{{n}_{aapoisum}}{{n}_{aatotalsum}} * \frac{{n}_{aasumpermoltotal}}{{n}_{aasumpermolpoi}} = {\%}_{aa} * \frac{{n}_{aasumpermoltotal}}{{n}_{aasumpermolpoi}}$$

signature comprising high performance liquid chromatography (HPLC) data, infrared spectroscopy (IR) data (i.e., using infrared radiation to assess vibrational modes arising from atoms within protein molecules and relating the vibrational modes to the structure of the protein), or other data. The AAA data may be real data collected from a biological sample, simulated AAA data, or a combination of real and simulated data. Real AAA data may be collected using one of many methods. For example, clinicians may perform hydrolysis on the protein sample to liberate amino acids from proteins, separate the amino acids from one another, and use one or more detection methods to quantify and label the amino acids in the sample. Simulated AAA data may be created by mixing previously-obtained sample data obtained from proteins from different sources (e.g., yeast, hen's eggs).

The AAA data may be analyzed or processed using one or more machine learning algorithms. For example, the AAA data may be analyzed or processed using a pseudo-Siamese neural network system with one or more layers of neurons. The system may compare a theoretical AAA for a POI against a heterogeneous sample to predict the POI %. The system may train this predicted POI % against a calculated POI % for the heterogeneous sample. Training may be performed over several epochs before testing commences. The system may reserve a portion of the training data for validation, to fine-tune the training process before finally testing on AAA data. In other embodiments, the AAA data may also be analyzed by non-neural network methods, including tree-based methods, logistic regressions, and support vector classifiers (SVCs). Because neural networks are generally more effective than these methods for determining the POI %, these additional machine learning algorithms may be used for applications which require less complexity, such as detecting whether a POI is in a biological sample.

Experiments using the system disclosed herein indicate that protein AAA fingerprints provide a reliable signal for not just protein presence but concentration estimation. The system herein not only offers robust multi-POI concentration estimation but may require less labor to employ given that the same AAA standards can be re-used across many POIs. Furthermore, through architectures encouraging zero-shot learning, neural networks trained on AAA can competently predict not just POIs identified at time of training but also POIs not used in training. The system may provide cost advantages by employing methods for building synthetic data from “real” data to reduce sample size requirements. The disclosed system may accelerate discovery operations, aid in the creation of calibration standards, reduce costs for early stage POIs, and increase executional confidence through another measure of purity.

### Proteins

Historical or available AAA results for this system may be obtained from a set of naturally occurring proteins occurring inside hen egg white and another set of host cell proteins. In some embodiments, the host cell is a plant cell or an animal cell. In embodiments, the host cell is a microbial cell, e.g., a bacterial cell or a fungal cell. The fungal cell may be a Pichia species, a Saccharomyces species, a Trichoderma species, or an Aspergillus species; a bacterial cell may be a Pseudomonas species or an E. coli species. The Pichia species may be Pichia pastoris or the Saccharomyces species is Saccharomyces cerevisiae. The sample may comprise a culturing medium for cultivating a host cell. The host cell may be a Pichia cell, a Saccharomyces cell, a Trichoderma cell, a Pseudomonas cell, an Aspergillus cell, or an E. coli cell. The Pichia cell may be a Pichia pastoris cell or the Saccharomyces cell may be a Saccharomyces cerevisiae cell. Information relating to the proteome of many species of host cells are described in publicly available databases and/or available in the scientific literature.

Naturally occurring proteins found in a hen egg white include ovalbumin, ovotransferrin, ovomucoid, ovoglobulin G2, ovoglobulin G3, lysozyme, ovoinhibitor, ovoglycoprotein, flavoprotein, ovomacroglobulin, ovostatin, cystatin, avidin, ovalbumin related protein X, and ovalbumin related protein Y.

### System and Method

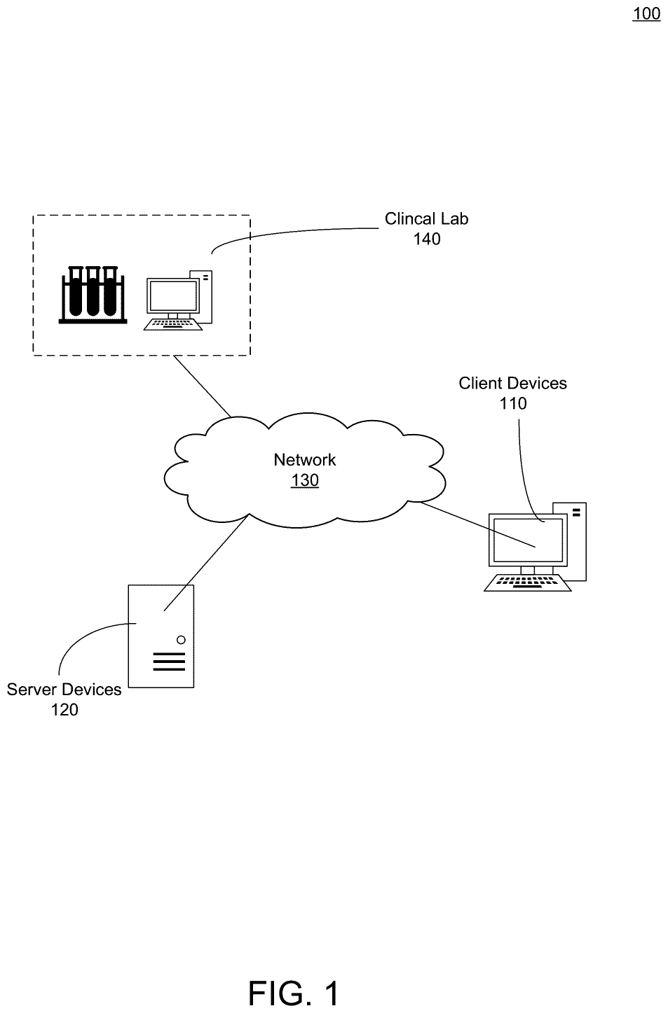

FIG. 1 illustrates an ecosystem 100 for determining a percentage of a protein of interest (POI %) in a biological sample. The ecosystem 100 may include a clinical lab 140, one or more client devices 110, and one or more server devices 120 connected by a network 130.

The clinical lab 140 may be one or more facilities used for producing AAA data for analysis. The AAA data may include simulated data, real data, or a combination of both. The clinical lab 140 may include one or more workstations providing reagents (e.g., buffer solutions, dyes), pipettes, droppers, reaction chambers, microplates, and other devices for processing one or more biological samples to create AAA data. The clinical lab 140 may also store collected AAA data, which may have been produced in the lab or may have been retrieved from other sources (e.g., other clinical lab 140s), that is used for creating synthetic AAA data. One or more components of the ecosystem may combine synthetic AAA data with real AAA data to produce datasets for machine learning processing. The clinical lab 140 may include one or more computer terminals to transport data to server devices 120 for machine learning processing or for other data processing tasks (e.g., data compression).

The clinical lab 140 may separate and quantify amino acids in samples using one or more of a number of techniques. For example, the clinical lab 140 may use paper chromatography, thin-layer chromatography, low-pressure ion-exchange chromatography, ion-exchange high performance liquid chromatography (HPLC), reversed-phase HPLC, gas chromatography, capillary electrophoresis, and mass spectrometry.

The client devices 110 may include computing devices for providing end users (e.g., clinicians, lab technicians, or food scientists) with access to analysis parameters and information produced by the ecosystem 100. For example, the client devices 110 may provide end users with calculated POI percentages for tested samples. Additionally, the client devices 110 may enable end users to adjust parameters for training and testing a machine learning model (e.g., adjusting a number of training epochs, changing a cost function, changing a type of machine learning algorithm, adjusting a split between training and validation data, changing an activation function, or changing another parameter). The client devices 110 may include computing devices such as laptops, mobile computing devices (e.g., smartphones, tablets), desktop computers, mainframe computers, terminals, or other computing devices.

The server devices 120 may include computing devices used for computer processing (including machine learning processing) and data storage tasks. The server devices 120 may receive AAA data from the clinical lab 140 or from other sources and may create datasets by mixing known AAA data to create simulated data, or by mixing simulated AAA data with real AAA data. The server devices 120 may store the AAA data in memory, such as RAM, ROM, flash memory, cloud storage, or other memory. The server devices 120 may implement one or more pre-processing tasks on the AAA data (e.g., data compression algorithms). The server devices 120 may perform machine learning tasks to process the AAA data, including implementing neural network 130s or other machine learning algorithms. The server devices 120 may provide products or outputs of the machine learning algorithms, including POI percentages, to the client devices 110 to be viewed by end users. In some instances, the server devices 120 may also include computing devices such as laptops, mobile computing devices (e.g., smartphones, tablets), desktop computers, mainframe computers, terminals, or other computing devices.

The network 130 may connect some or all of the components of the ecosystem to one another. The network 130 may be an Internet network 130, a MAN, a LAN, a WAN, a Wi-Fi network, a cellular network, or another network. The network 130 may enable the ecosystem to be fully connected.

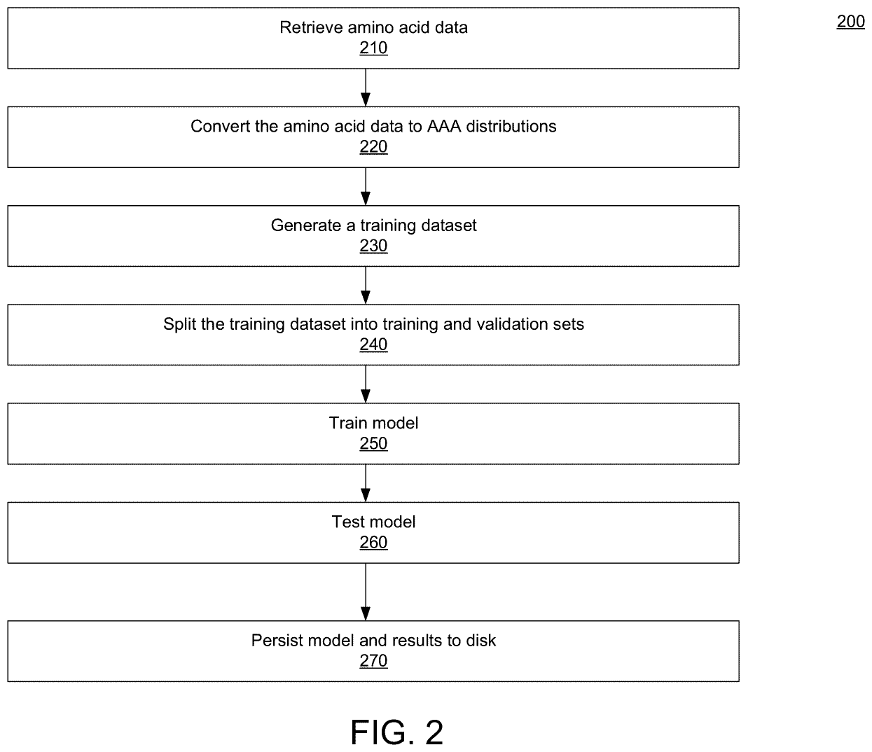

FIG. 2 illustrates a pipeline 200 for implementing a machine learning algorithm on a set of AAA data to determine a percentage of a protein of interest. The pipeline 200 illustrates the steps of training, validating, and finally testing the AAA data. One or more components of the ecosystem (e.g., server devices 120 or client devices 110) may use training and validation to create a machine learning model that may be applied generally to AAA data to produce accurate POI % predictions. For brevity, the aforementioned one or more components may be referred to herein interchangeably as “the system.” The system may perform validation alongside training, while testing may be performed following completion of several epochs of training.

The system may analyze real AAA data determined from biological samples, simulated AAA data, or a combination. In a first operation 210, the system may receive amino acid sequences from samples as, for example, FASTA files received from the clinical lab. In a second operation 220, the system may convert the amino acid data to AAA distributions. The simulated AAA data may be a mixture of known amino acid compositions of proteins. In one embodiment, a distribution of simulated AAA data may include a composition of amino acids from 19 “expected” proteins, 21 egg proteins, and 6049 yeast proteins. The system may create the simulated mixture by concatenating amino acid sequences in amino acid sequence files. This heterogeneous AAA distribution may simulate an impure sample of one of the 19 “expected” proteins and may range from 10-99% purity. The heterogeneous AAA distribution may combine theoretical 100% purity AAA values of different proteins, where each protein's concentration may be different from the others. In some embodiments, these components of the “observed” AAA may be weighted, with their combination comprising a weighted average. Multiple such AAA distributions may comprise an “observed” set. The system may also retain a set of “theoretical” homogeneous (100% purity) AAA distributions for each of the expected proteins. In a third operation 230, the system may generate a training dataset from these distributions. In a fourth operation 240, the system may split the training dataset into training, validation, and test sets. For example, the system may reserve 70% of the data for training, 15% for validation, and 15% for testing.

In a fifth operation 250, the system trains the model. For a particular protein of interest, the system may train the model by comparing, for a particular protein of interest, the theoretical AAA distribution of the protein of interest to the set of “observed” AAA distributions. In some embodiments, a pseudo-Siamese neural network may process the theoretical distribution and an observed distribution and produce a result POI % for the protein of interest. The pseudo-Siamese neural network may include a pair of concatenated input vectors without corresponding parallel branches. The pair of concatenated input vectors may comprise a first input vector comprising of historical or available AAA results and a second input vector comprising theoretical AAA results at 100% purity. To determine the accuracy of this result POI %, the machine learning model may compare the result to a calculated POI % for the expected protein in the observed distribution. This calculated POI % may be determined using high performance liquid chromatography (HPLC) or by another method. The comparison function of the neural network may be automatically learned without external human input or intervention. After the model calculates the error from this comparison, the error may be backpropagated through the model in order to change the model weights, ideally reducing the error in successive iterations. For all of the expected proteins, the system may successively perform training until the machine learning model can be used to predict generally accurate POI percentages for any input protein. In some embodiments, training of the model may be performed in one hour or less.

As the model is trained, it may be validated using some of the data from the training set. This validation data may be processed simultaneously with the training data in order to fine-tune the machine learning model. But unlike the training data, the model may not backpropagate error resulting from processing the validation data. Instead, the validation using the validation data may be treated as a preliminary test of the model and may be performed until results are robust enough to test the final model.

The model may be implemented using one of a variety of machine learning algorithms. For example, the system may implement the model using a support vector classifier (SVC), a classification and regression tree (CART), Adaboost, a logistic regression, or another method. The system may also implement the machine learning model using a neural network, such as a convolutional neural network (CNN), a recurrent neural network (RNN), or another neural network. The system may use a deep neural network, with multiple layers of neurons. In some embodiments, the neural network has one, two, three, or four inner hidden layers. For example, a one-layer neural network may use eight neurons. A two-layer neural network may have a first layer of 16 neurons and a second layer of 8 neurons. A three-layer neural network may have a first layer of 32 neurons, a second layer of 16 neurons, and a third layer of 8 neurons. A four-layer neural network may have an input layer, three inner hidden layers of 64, 32, 16, and 8 neurons, and an output layer. The machine learning model may produce a prediction using an output classifier layer. A binary classifier layer may output a number between 0 and 1 to indicate a predicted POI %. The output layer may also be a multiclass classifier, producing POI % predictions for multiple proteins of interest. In some embodiments, the machine learning model may predict whether there is a presence of a particular POI. In these embodiments, the output layer may be a sigmoid layer, and may output numbers either close to 1 or 0. Numbers close to 1 may indicate high likelihoods of the POI being present, while numbers close to 0 indicate low likelihoods.

In a sixth operation 260, the system tests the model. The model may be tested using AAA data it was not exposed to during training. When the model produces a prediction, the prediction may be provided to end users via end user client devices. The AAA data used for training, validation, and testing may be unlabeled data. Because the model need not require labeled data to predict a POI %, the model may be tested on proteins outside of the expected protein set.

This may make the model similar to a zero-shot model, as it may be able to provide POI % estimates for proteins it had not encountered during training. Notably, implementing the model may not require learning of a lower dimensional representation.

In a seventh operation 270, the system may persist/store the model and the test results to disk.

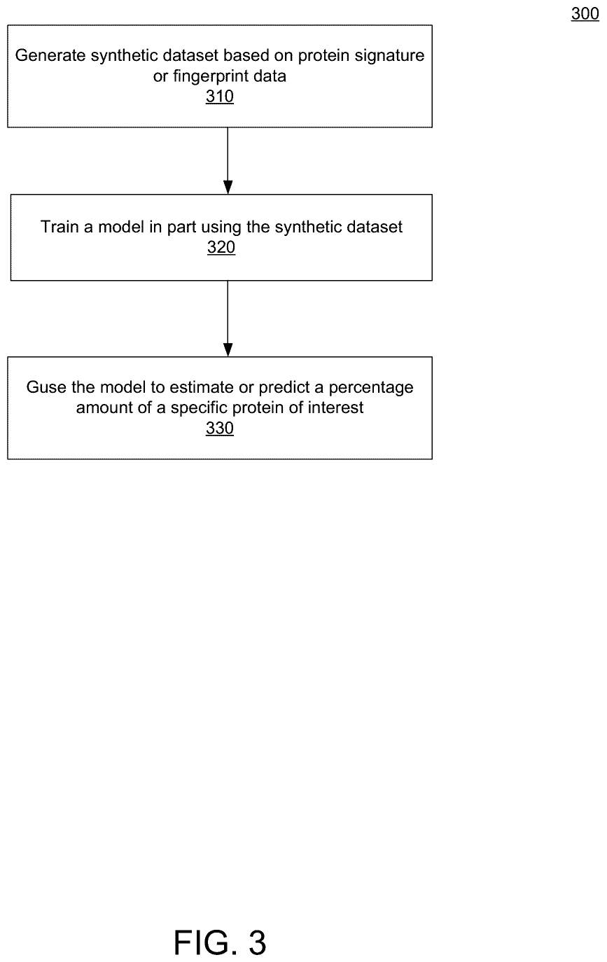

FIG. 3 illustrates a process flow diagram 300 for predicting a POI % from AAA data. The AAA data may be produced in a clinical lab, synthesized, or may be a combination of both. A machine learning model processes the AAA data to produce the POI % prediction.

In a first operation 310, the system generates a synthetic dataset based on protein signature or fingerprint data. The system may combine real and synthetic AAA data. The system may then split the dataset into training and validation sets. The dataset may include theoretical AAA distributions for expected proteins and “observed” AAA values comprising heterogeneous amino acid distributions from a variety of proteins in a sample.

In a second operation 320, the system trains a model in part using the synthetic dataset. The proteins are not required to be labeled during training. The model may be trained to compare a theoretical AAA distribution for a POI to a heterogeneous observed AAA distribution containing the POI at a purity between 10-99%. The model may compare a predicted POI % against a calculated POI % for the observed AAA distribution and may iterate training until the model is able to generally make robust predictions. The system may perform validation alongside training, using data set aside for validation purposes. While training, the model may backpropagate error between the calculated POI % and the predicted POI %. But during validation, the model may not backpropagate the error.

In a third operation 330, the system uses the trained model to estimate or predict a percentage amount of a specific protein of interest (POI %) in one or more heterogeneous samples. The model may receive an unlabeled observed test AAA distribution along with a theoretical AAA distribution for a protein of interest. The model may then produce a prediction from these inputs to predict a POI %. The POI % may be provided to one or more client devices of end users. The system may predict one or more POI % in one or more heterogeneous samples.

### Experimental Evaluation of the System

Disclosed are methods for evaluating that 1) that it is unlikely that two proteins have the same AAA distributions, enabling the prediction of POI % from AAA distributions and 2) the system disclosed has robust predictive power for POI percentages between 10% and 99%.

The challenge set is a set of proteins against which a POI may be confused (having a too similar amino acid distribution to be detected separately). With proteins from the challenge set, the system can accurately predict POI % from AAA distributions because it is unlikely that two proteins will have the same AAA distributions. This may be demonstrated using the following experiment. 1000 (n %) objects each representing 0.1% of the mole % in a AAA distribution. Those items can be “assigned” into 22 groups (raa) which correspond to an amino acid. The AAA distribution then may become the mole % for each amino acid having assigned those 1000 items. The following equations illustrate the probability of two proteins yielding the same AAA results or, in other words, the chance that the 1000 objects end up in the same bins for two proteins. Performing these calculations assumes that amino acid distributions are spread uniformly across the AAA space (any given configuration of the 1000 objects into the bins is equally likely as another).

The following formulas arrive at solutions for the above two probabilities starting with the problem reduction to identical objects assigned to distinct bins:

$$Unique arrangements of identical objects into distinct bins .$$

$${n}_{distinct} = ( \begin{matrix}{n}_{\%} + {r}_{aa} - 1 \\ {r}_{aa} - 1\end{matrix} ) = \frac{( {n}_{\%} + {r}_{aa} - 1 ) !}{( {r}_{aa} - 1 ) ! ( {n}_{\%} + {r}_{aa} - 1 - {r}_{aa} + 1 ) !} \qquad Equation 1$$

Turning this into a probability of collision for a POI against a single challenge protein:

$$Probability of target protein with same AAA fingerprint as challenge .$$

$${p}_{collision} ( {P}_{POI} , {P}_{challenge} ) = \frac{1}{{n}_{distinct}} = \frac{( {r}_{aa} - 1 ) ! ( {n}_{\%} + {r}_{aa} - 1 - {r}_{aa} + 1 ) !}{( {n}_{\%} + {r}_{aa} - 1 ) !} \qquad Equation 2$$

With some simplification, one arrives at the following:

$$Simplified probability of POI with same AAA fingerprint as challenge .$$

$${p}_{collision} ( {P}_{POI} , {P}_{challenge} ) = \frac{{n}_{\%} ! ( {r}_{aa} - 1 ) !}{( {n}_{\%} + {r}_{aa} - 1 ) !} \qquad Equation 3$$

Given this formulation, the chance of no collision becomes:

$$Probability of target protein with same AAA fingerprint as challenge .$$

$${p}_{nocollision} ( {P}_{POI} , {P}_{challenge} ) = 1 - \frac{{n}_{\%} ! ( {r}_{aa} - 1 ) !}{( {n}_{\%} + {r}_{aa} - 1 ) !} \qquad Equation 4$$

Cardinalities of the sets can describe the chance of at least one collision for a POI against any protein in a challenge set (S_{challenge}):

$$Probability of target protein with collision in challenge set .$$

$${p}_{onecollision} ( {P}_{POI} , {S}_{challenge} ) = 1 - {( 1 - \frac{{n}_{\%} ! ( {r}_{aa} - 1 ) !}{( {n}_{\%} + {r}_{aa} - 1 ) !} )}^{\vert {S}_{challenge} \vert} \qquad Equation 5$$

The following equation estimates the number of collisions for an individual protein:

$$Number of expected collisions for a single POI against a set .$$

$${n}_{collision} ( {P}_{POI} , {S}_{challenge} ) = ( \frac{{n}_{\%} ! ( {r}_{aa} - 1 ) !}{( {n}_{\%} + {r}_{aa} - 1 ) !} ) * \vert {S}_{challenge} \vert \qquad Equation 6$$

The following equation calculates total collisions in a set:

$$Number of total mutual collisions in a challenge set .$$

$${n}_{collision} ( {S}_{challenge} ) = ( \frac{{n}_{\%} ! ( {r}_{aa} - 1 ) !}{( {n}_{\%} + {r}_{aa} - 1 ) !} ) * ( {\vert {S}_{challenge} \vert}^{2} - \vert {S}_{challenge} \vert ) \qquad Equation 7$$

These are evaluated below in the results section using the actual cardinalities from the challenge sets above. As described below, this method does not consider inaccuracies in AAA beyond the 0.1% precision.

### Simulation Method

AAA fingerprints likely do not uniformly distribute with some proteins being more likely to have amino acid compositions similar to those seen in other related proteins. This could make the analytical solution yield an under-estimate of collision probabilities, especially for “family” collision counts where a challenge set contains many similar proteins. AAA distributions of actual proteins may be used to evaluate similarities of fingerprints.

Simulations using the individual theoretical AAA evaluate both any collisions within the set (“family collision rate”) and then collisions for a target POI across all proteins in all sets (“individual collision rate”). In the case of individual collision rate challenging a POI against all challenge sets, each of the 19 proteins in the “expected” proteins challenge set above is used individually as the POI to determine resilience of the fingerprints across multiple proteins of interest. More precisely, analysis first searches for matching fingerprints between all unique proteins within a single challenge set using those proteins' theoretical AAA results at 100% purity (“family”) before then using 19 different POIs to simulate how many times a collision is found between that example POI and any protein in any challenge set (“individual”).

Each “scenario” may involve applying Gaussian noise to the theoretical pure AAA results (at the amino acid level) for the POI and challenge sets where μ=0 and σ is a multiple of 0.2% up to 5.0%. Based on historical AAA results, the study may assume measurements reported to 0.1% from AAA. This may simulate error in the AAA itself and fingerprint's resilience to noise.

### POI Prediction

The disclosed system may determine whether modeling can detect the POI fingerprint in a very noisy environment (assuming ≥50% POI concentration) given only the AAA results.

The system may use multiple types of classifiers through comparison of validation performance (logistic regression, SVC, single CART tree, Adaboost, random forest). A 70%/15%/15% split for training, validation, and test may be used.

### Dataset Generation for POI Prediction

In one embodiment, the system may generate a set of 10,000 theoretical AAA samples using combined challenge sets with a sample POI from “expected” proteins. Specifically, a (uniform) random number generator may decide the POI % for a sample (limited to 50 to 99%) and then the rest of the sample is simulated as a random set of proteins across all three challenge sets in random proportions. The simulation then may generate the overall AAA by mixing each proteins' theoretical AAA distributions as a weighted average with weights equal to simulated protein proportion. Each of the 19 proteins in the “expected” set may be chosen as the POI randomly per simulated sample but all other proteins across all challenge sets (n=6089) may be used randomly to build the rest of the AAA result. This may simulate a highly heterogeneous environment.

### Purity Estimation

The system may attempt to build a regressor which estimates the POI purity as a percentage (% purity) given only the sample AAA and the POI' s theoretical 100% purity AAA results. This first formulation assumes the POI at concentrations greater than or equal to 50%.

### Dataset Generation

The system may train a model using actual observed AAA and the theoretical AAA for the POI against POI % calculated using accepted methods like AAA and HPLC (for proteins with high confidence calibration). To create a set to train against, the system may also generate a training set of 10,000 theoretical AAA results using the method described above, once more restricting the POI % to 50-99%. The inputs then become the simulated AAA mole % values for the heterogenous sample. The system may record the randomly chosen POI % for each generated AAA from the dataset generation step but does not provide that percentage or POI name to the model as input.

### Neural Network Topology

In addition to the methods explored for POI prediction, a neural network may act as a regressor to predict POI % (known from dataset generation but kept hidden from the model). This regressor may use both the AAA mole % values resulting from the weighted average described above and the theoretical AAA distribution of the POI at 100% purity. Note that, while candidate neural networks may be given access to both the POI theoretical distribution and the observed distribution, those models may not be given the names of the POIs.

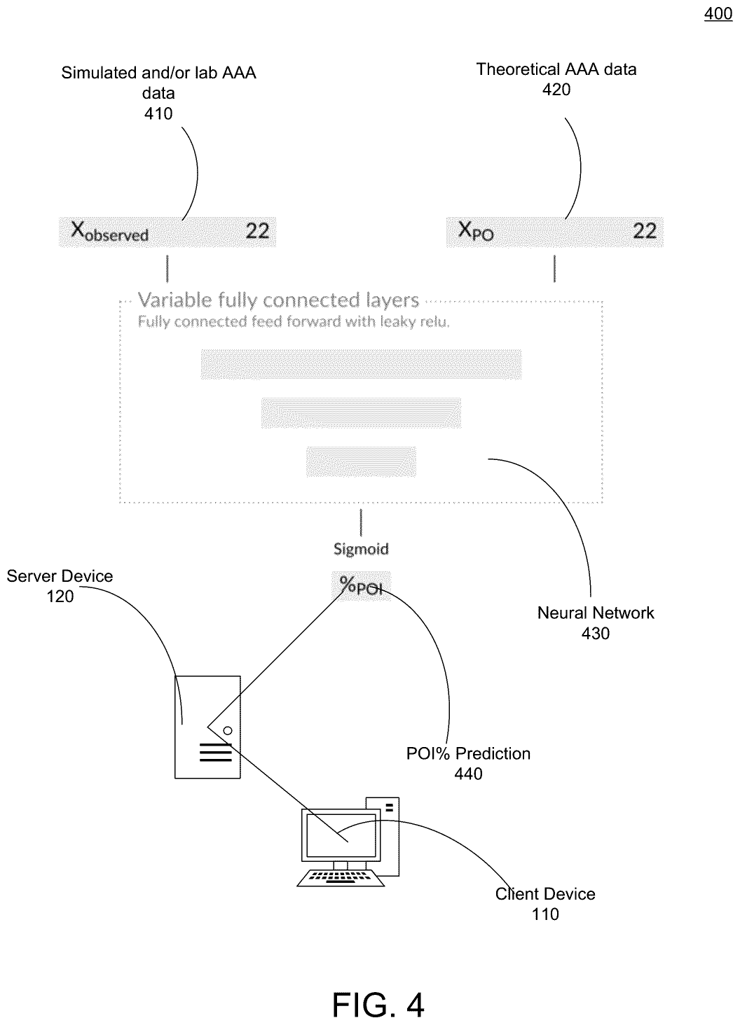

FIG. 4 illustrates an example neural network topology 400. The example topology takes input vectors 410 and 420 and processes them using neural network 430. The output POI % prediction 440 may be provided to the server device 120, which may provide the prediction to a client device 110 of an end user. Neural network 430 may use a limited sweep to fit a regressor using all combinations of different levels of L2 (ridge) regularization at (0, 0.1, 0.2, 0.3, 0.4) and the following sets of fully connected inner hidden layers:
Single layer of 8 neurons.
Two layers: 16 and 8 neurons in decreasing order.
Three layers: 32, 16, and 8 in decreasing order. Note that this study uses Adam and mean squared error loss.
Four layers: 64, 32, 16, and 8 in decreasing order.

In one embodiment, the system may use 20,000 AAA results with the same method above but may allow any concentration (e.g., extending to concentrations under 50%) of the POI to investigate performance with a POI below 50% concentration (specifically 10% to 99%). Another sweep of the same parameters for a neural network-based regressor may investigate if the same topology and configuration remain preferred from the purity estimation for concentration estimation.

### Evaluation Results

Given the formulations above which assume roughly even distribution of AAA mole % vectors, the AAA data represents a vast space with the probability of any two proteins encountering the same AAA “fingerprint” around 4*10⁻⁴⁴ with rₐₐ=22. Indeed, under the same assumption, the probability of a collision for a POI against any other protein in any of the challenge sets remains still far less than one millionth of one percent (2*10⁻⁴⁰). No mutual collisions are expected on average for any of the challenge sets when comparing one protein against the rest. Therefore, the fingerprint could uniquely identify a protein.

### Simulation Results

The analytical solution assumes a uniform distribution of fingerprints which, in practice, could prove unlikely. Therefore, a simulation may both explore family-wise collisions (total number of collisions between all proteins but only within in a single challenge set) and collisions with a POI against all challenge sets.

Starting with the family-wise collision count, the simulations show no collisions in either the “expected” or “hen” sets and 69 collisions in the “yeast” set. In other words, about 98.9% of proteins see no “family” collision.

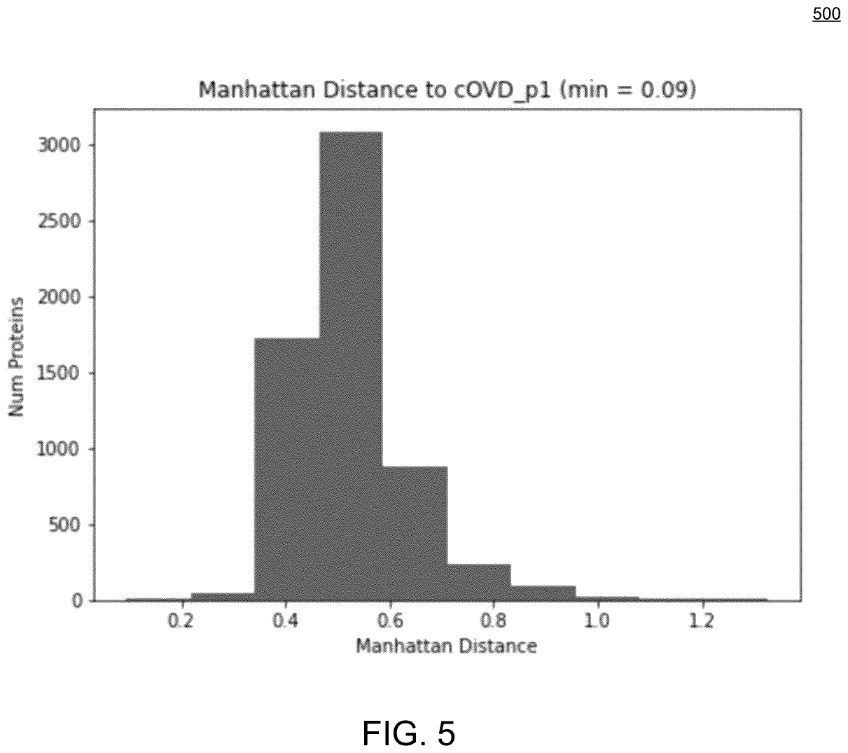

Next, the simulation uses each of the 19 proteins in the “expected” set as a POI, looking for collisions against any other protein in any challenge set at 1% Gaussian noise. In all cases, this study finds no collisions within the simulation. FIG. 5 illustrates an analysis 500 of a likelihood that two proteins will have the same AAA signature. Analysis finds large distance between one of these POIs and all the other proteins with a minimum Manhattan distance of 9 points (0.09).

These results remain unchanged when increasing noise to σ=5%, indicating that, except for some localized clusters in the “yeast” dataset, the theoretical AAA show substantial distance between most proteins. Regardless, assuming only a few thousand potential proteins present and similar distance between protein fingerprints, the simulations add further evidence that AAA fingerprints uniquely identify distinct proteins.

### POI Prediction

For predicting a presence of a POI in a sample, Adaboost at 20 estimators with a max depth of 5 per estimator for predicting the POI from the AAA mole % distribution yields robust performance. The system produces accuracy above 99% across test, train, and validation sets. This indicates resilience to overfitting and that the POI fingerprint remains detectable in highly heterogeneous environments with noise assuming POI concentration of at least 50%.

### Purity Estimation

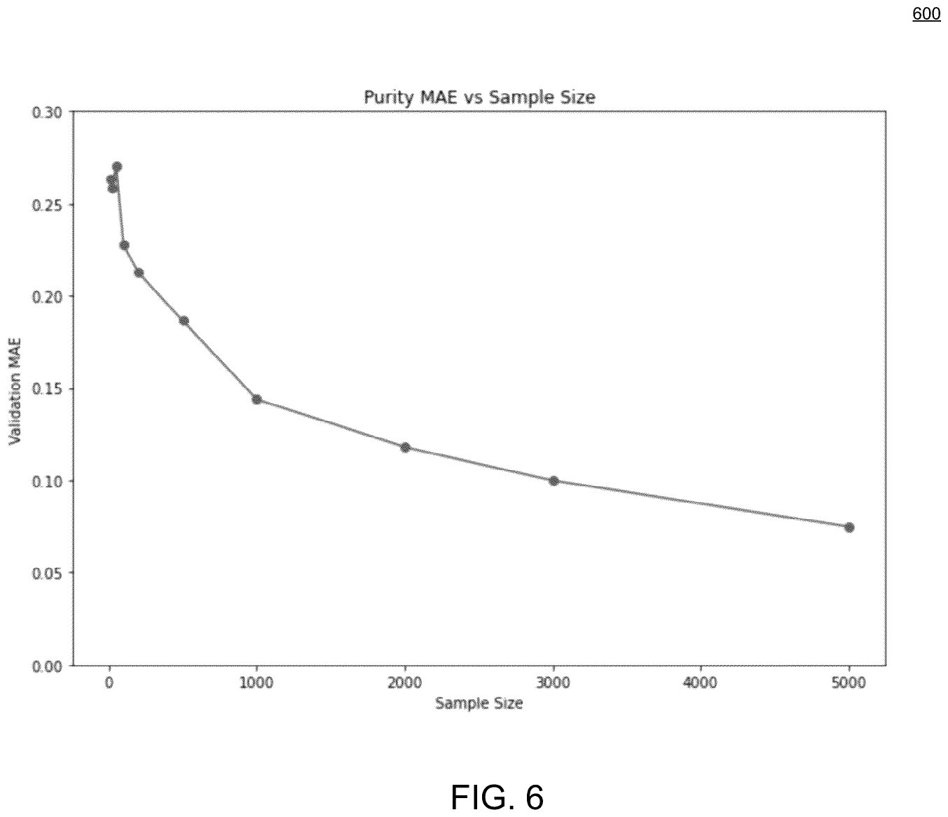

FIG. 6 illustrates the efficacy chart 600 of the system at predicting a POI %. Again using validation set MSE, this study uses a neural network with inner layers of 32, 16, and 8 neurons regularized with L2 at 0.2. This model provides improvements over other non-neural approaches as discussed below. After 20 epochs of training, mean absolute error (MAE) settles around 2.7%. For a test set, the same model configuration performs at 2.8% test mean absolute error, indicating that the model can predict POI % with an error of about 3 points in novel data. While the simulated AAA offer substantial noise, the POI % is simulated to be 50-99%. The investigation shows that an MAE of under 20% becomes possible around 500 samples and an MAE under 10% at around 3,000.

A sample size of 3,000 could prove costly. However, implementation may find that simulated data perform well in making predictions in “real world” data. In practice, a mixture of “real” observed AAA data and “synthetic” data from simulations may provide a good compromise on cost to achieve high performance. Regardless, performance at 3% offers strong evidence for system effectiveness.

### Concentration Estimation

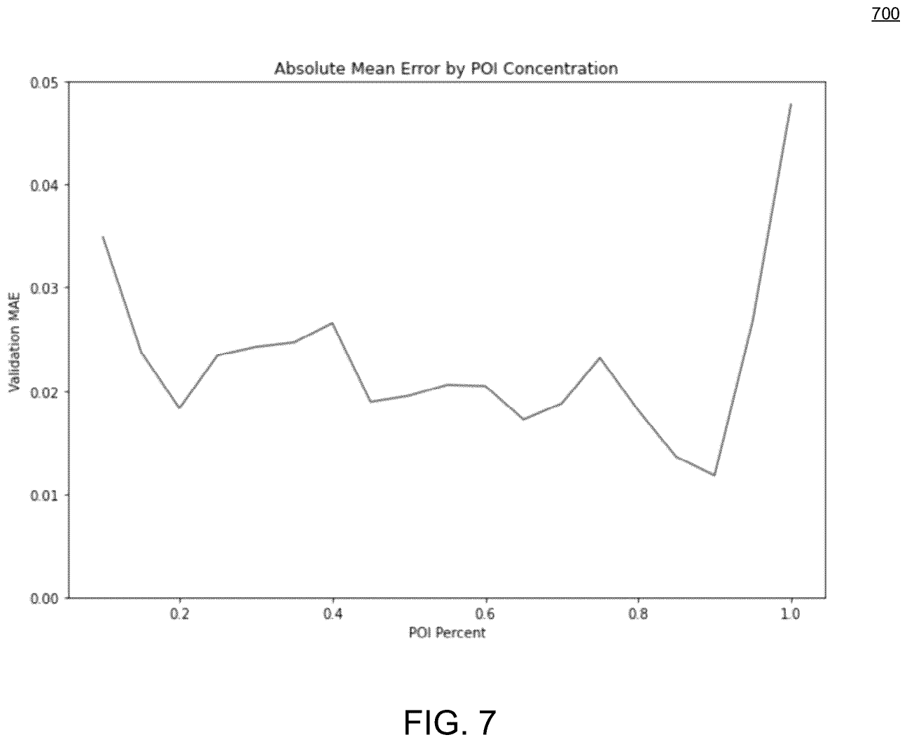

FIG. 7 illustrates results 700 of an experiment examining model performance when the POI does not necessarily constitute a majority of the sample. Like in purity estimation, comparison on validation set performance provides for inner layers of 32, 16, and 8 neurons but with an L2 (ridge) regularization of 0.1. Through trained with 20,000 theoretical AAAs, post-hoc analysis finds an MAE under 20% at 1,000 samples and under 10% at 3,000, the latter of which tracks closely to purity estimation results. That said, performance varies depending on the “actual” POI concentration.

The bowl-like shape of the performance may indicate the neural network biasing against extreme results (under 20% concentration or over 90%). Even still, note that MAE remains under 5% in all cases, lending evidence towards the fourth hypothesis.

### Zero-Shot Performance

While double the MAE for proteins seen in training as the POI, post-hoc experimentation still achieves a 6% test set mean absolute error for proteins unseen in training. This indicates the model in its current form could contribute to zero shot learning. Use of the validation set changed L2 regularization to 0.3. Interestingly, if only the “expected set” proteins are used as POIs in training, the MAE increases to 16%, indicating that a large diversity of POIs in training prevents overfitting. Regardless, these results indicate that the described model can provide concentration estimation for “out of sample” proteins for which the model is not explicitly trained. This enables both prediction of POI % for primary POIs but also other proteins in ad-hoc analysis like in looking for specific contaminants. In other words, one model understanding AAA data in general could apply to specific POIs without retraining.

### Discussion of Results

These results indicate the system may have an ability to predict arbitrary POI concentration using only AAA data without protein-specific calibration, solving an important problem that reduces costs and increases execution speed.

Methods for protein concentration estimation require time and effort to establish, often through protein-specific calibration. However, as the AAA data may not require protein-specific standards, the described models provide a concentration estimation tool without requiring many protein-specific assumptions or labor. Availability of this additional low-assumption metric may improve confidence in quality control systems, may provide a mechanism to check or improve other protein concentration estimation methods, and may offer a clean signal for optimization when working with POIs. Furthermore, in the early stages of work with a protein or in ad-hoc analysis of certain POIs (such as specific contaminants), this model could allow research teams to skip protein specific calibration for concentration estimation altogether, accelerating discovery and analysis while reducing or delaying costs in the form of less labor. Taken together, the disclosed system may enable more confident execution at higher speeds and lower expense.

### Comparison to Other Methods

### Sample Size and Real Data

The methods disclosed herein could be used to enhance predictive power of machine learning algorithms by augmenting real sample data with simulated data. Purity and concentration regression mean absolute error drops below 10% at around 3,000 samples. Compiling such an archive could prove prohibitively expensive and time consuming. However, the use of simulated data provides an opportunity to use a small number of samples with noise for data augmentation to build the required sample size in conjunction with theoretical data.

### Extreme Concentrations

The results slight show some degradation in regression performance when predicting POI % close to edge of acceptable values (10% and 99%). While the degradation is not substantial, application of instance weights at the edges could potentially reduce this bias. Furthermore, experiments at below 10% POI could extend the model performance for very low concentrations.

### Comparison to Non-Neural Models

Of the non-neural models, support vector machines (SVM) saw the best validation set performance for POI % estimation. However, it still achieves around 10% MAE so the neural network remains the preferred approach.

| Model | Best Training MAE | Best Validation MAE |
|---|---|---|
| Linear (Lasso) | 0.26 | 0.26 |
| SVM | 0.12 | 0.12 |
| Tree | 0.24 | 0.24 |
| Adaboost | 0.24 | 0.24 |
| Random Forest | 0.24 | 0.23 |
| Neural Network | 0.01 | 0.03 |

### Machine Learning

In the embodiments described herein, the system uses machine learning to analyze amino acid composition of a sample containing one or more proteins. The machine learning algorithm may be a supervised machine learning algorithm. The supervised machine learning algorithm may be trained on time-series amino acid composition data. For example, amino acid composition data from a first time may be completely synthetic, but may be updated at a second time to include AAA results obtained from a lab. The composition of the AAA data may change over time to reflect additions of synthetic or lab-obtained AAA components. The supervised machine learning algorithm may be a regression algorithm, a support vector machine, a decision tree, a neural network, or the like. In cases in which the machine learning algorithm is a regression algorithm, the weights may be regression parameters. The supervised machine learning algorithm may be a binary classifier that predicts a percentage of a protein of interest (POI) in the sample. The binary classifier may generate a POI % between 0 and 1. Alternatively, the supervised machine learning algorithm may be a multi-class classifier that produces predictions for multiple POI percentages.

### Neural Networks

The present disclosure describes the use of machine learning algorithms to predict POI percentages of various proteins. The machine learning algorithms may be neural networks. Neural networks may employ multiple layers of operations to predict one or more outputs (e.g., a risk score) from one or more inputs (e.g., health measurement and socioeconomic data). Neural networks may include one or more hidden layers situated between an input layer and an output layer. The output of each layer can be used as input to another layer, e.g., the next hidden layer or the output layer. Each layer of a neural network may specify one or more transformation operations to be performed on input to the layer. Such transformation operations may be referred to as neurons. The output of a particular neuron may be a weighted sum of the inputs to the neuron, adjusted with a bias and multiplied by an activation function, e.g., a rectified linear unit (ReLU) or a sigmoid function. The output layer of a neural network may be a softmax layer that is configured to generate a probability distribution over two or more output classes.

Training a neural network may involve providing inputs to the untrained neural network to generate predicted outputs, comparing the predicted outputs to expected outputs, and updating the algorithm's weights and biases to account for the difference between the predicted outputs and the expected outputs. Specifically, a cost function may be used to calculate a difference between the predicted outputs and the expected outputs. By computing the derivative of the cost function with respect to the weights and biases of the network, the weights and biases may be iteratively adjusted over multiple cycles to minimize the cost function. Training may be complete when the predicted outputs satisfy a convergence condition, e.g., a small magnitude of calculated cost as determined by the cost function.

### AAA Fingerprint Modeling Refinement for Low Cardinality Samples

While some applications of the systems and methods disclosed herein target environments with many proteins, many samples have relatively few proteins, thus operating in a slightly more difficult “low cardinality” environment. The methods disclosed herein may be modified to be more effective in low cardinality environments. The following experiment describes modifications to the model that may enable it to perform more effectively in these environments.

After employing a limited parameter sweep to optimize the AAA fingerprint method, one may implement an L2 regularization of 0.01, learning rate of 0.0001 (Adam), and four layers (64, 32, 16, 8) for samples which contain relatively few unique proteins, demonstrating results with samples between 2-30 proteins generated at uniform random distribution. This method may achieve around 6 point mean absolute error in a hidden test set comprised of these more difficult samples. Meanwhile, in a low heterogeneity environment, the zero shot performance may see 8 point mean absolute error. Experimental data finds an inverse relationship between mean absolute error and actual POI concentration with highest performance at highest concentrations both for “known” and zero shot proteins. These findings may disclose means to present lower confidence protein % estimations to end users.

To evaluate purity, this experiment uses a neural-network based model on amino acid analysis (AAA) data to estimate concentration or, in other words, how much of the total protein in a sample comes from a protein of interest (POI). This AAA fingerprint method may evaluate model performance under varying degrees of heterogeneity (how many different proteins are in a sample) and concentration (true POI %) using simulated data. Furthermore, the earlier mentioned performance, which uses low hundreds of samples, may be much larger than the typical number of unique proteins expected in samples associated with embodiments of the current disclosure.

Simulated data may be used to perform an additional limited sweep to evaluate expected performance and preferred parameters in samples with few proteins. These samples are called “low cardinality” due to the small number of unique proteins present.

This experiment evaluates model characteristics in lower cardinality samples, both for “known” proteins and the zero shot case.

### Known Proteins

Starting with proteins used in training, this experiment may re-run a limited parameter sweep using different number of inner layers (one to four layers of size 64, 32, 16, and 8 neurons), L2 regularization (0, 0.1, 0.01), and learning rates (0.01, 0.001). Using a dataset of 110,000 instances, one may choose a configuration based on best performance on a validation set (80% train, 10% validation split). One may compare results observed in the “best model” versus the performance at the a method comprising recommended levels1 (L2 of 0.1 and three layers of 32, 16, 8). One may also consider overall performance against a hidden test set of 10% before paper evaluating performance across different simulated concentration amounts.

### Computational Efficiency

Results for such an experiment may be obtained using a maximum of 30 unique proteins per sample. This experiment and production systems generate data points for the same POI and confounding protein mix at 20 concentrations each at multiples of 5% plus a random offset between 0 and 4% instead of just one data point per POI/confounding mix pair. This means one data point per POI/confounding mix between 0-4%, one between 5-9%, and so on. This may reduce time in data generation. Therefore, this study generates 5,500 POI/confounding set pairs before generating 20 data points for each pair at different POI % levels.

### Zero Shot Learning

In addition to testing the model on proteins used in training, this experiment may also investigate performance on “novel” proteins in the zero shot learning case. To investigate likely performance in a sample, this experiment uses the full observed AAA distribution from a particular sample as the “confounding” set and then uses goose OVL (“gOVL”) as the POI. Specifically, this experiment may generate 100 samples at 1% increments from 1% to 100% gOVL.

Other experiments may use a learning rate of 0.001 on Adam.

### Results

This experiment examines the sweep results before post-hoc investigation of performance across different concentration amounts within the hidden test set.

### Sweep Results

One configuration yields a mean absolute error of 9 points on the low heterogeneity validation set at 50 epochs. The sweep may suggest L2 regularization of 0.01, a learning rate of 0.001, and four layers (64, 32, 16, 8) with a validation set mean absolute error of 7 points also at 50 epochs. That being said, a learning rate of 0.001 may show “jumpiness” in loss when trained for longer so this experiment suggests a learning rate of 0.0001. Taken together, these parameters with the lower learning rate show a hidden test set performance of 5.5 points at 100 epochs, nearing original expected performance (3 points) from historical data. Of course, while not a focus of investigation for this experiment, the earlier mentioned lower error may suggest higher model performance in higher cardinality.

### Known Protein Performance

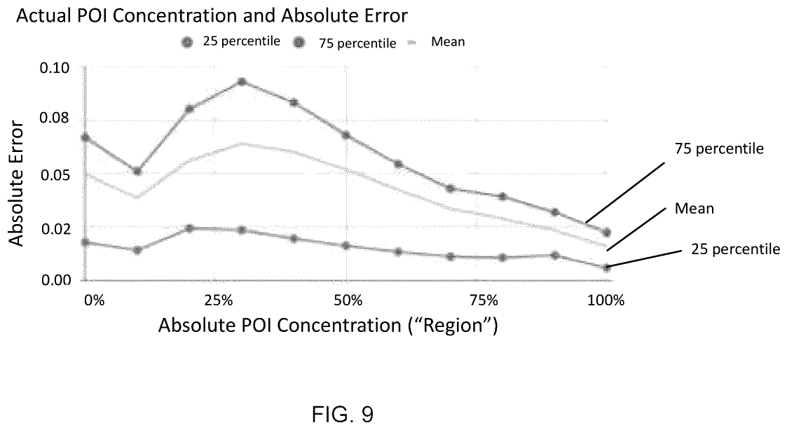

With the above sweep's model in place, examination of mean absolute error (FIG. 9) shows an inverse relationship between error and POI concentration for proteins seen in training.

This experiment may use 100 epochs to evaluate expected performance on unseen data because it is the training duration used in production.

Although the model never sees a region with over 10 points error at the 75th percentile, the model tends to over-estimate POI % in lower regions where its error is also the largest.

### Zero Shot Performance

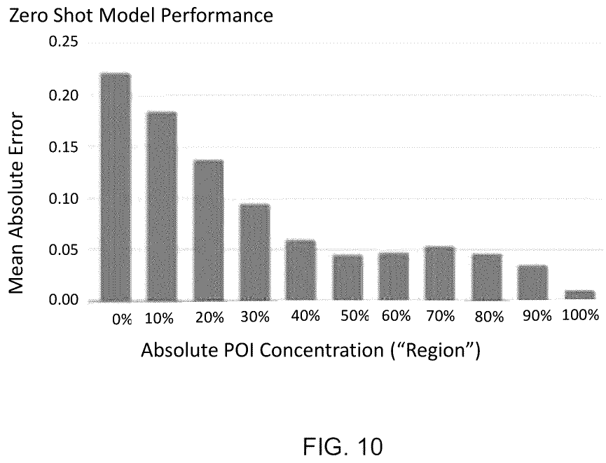

This experiment observes a mean absolute error of 8 points for “unseen” proteins (FIG. 10), similar to the 6 points observed in another method. Like for “known” proteins, zero shot sees an inverse relationship between POI % and error. Notably, the absolute error generally stays below 10 points above 30% concentration and 5 points beyond 50% concentration.

### Conclusion

This experiment recommends the following production parameters for low cardinality environments:
L2 regularization of 0.01
Learning rate of 0.0001 (Adam)
Four hidden layers of 64, 32, 16, 8 neurons.

Lower concentration regions may present challenges to use of the AAA fingerprint model. A prediction of 20% could be anywhere from 12% to 28% according to the above data at the 75th percentile of error. Therefore, one could use this model for detecting small concentration contaminants but, due to the magnitude and typical direction of the error, users of the model may consider reporting <30%, <20%, and <10% instead of the actual values under 30%. That in mind, the results from the zero shot case suggest particular caution when applying the model to previously unseen proteins under 30%. Of course, future work may consider instance weighting near the edges to attempt to encourage more learning in low concentrations.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

### Computer Systems

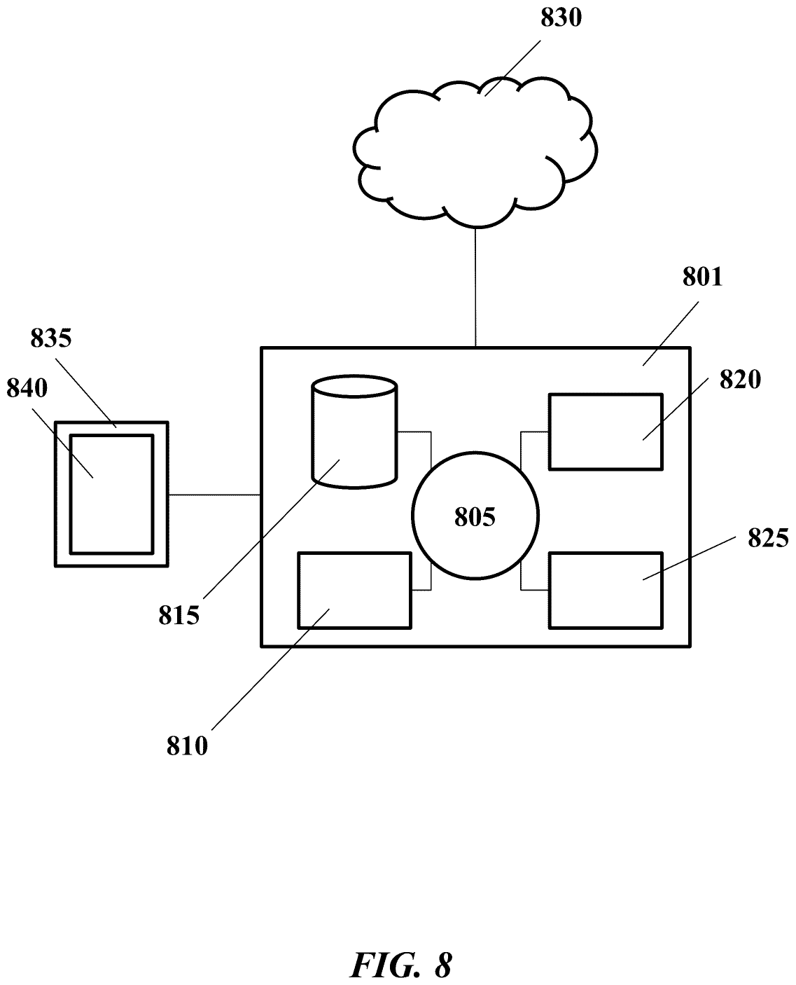

The present disclosure provides computer systems that are programmed to implement methods of the disclosure. FIG. 8 shows a computer system 801 that is programmed or otherwise configured to predict a POI %. The computer system 801 can regulate various aspects of machine learning analysis of the present disclosure, such as, for example, implementing a neural network. The computer system 801 can be an electronic device of a user or a computer system that is remotely located with respect to the electronic device. The electronic device can be a mobile electronic device.

The computer system 801 includes a central processing unit (CPU, also “processor” and “computer processor” herein) 805, which can be a single core or multi core processor, or a plurality of processors for parallel processing. The computer system 801 also includes memory or memory location 810 (e.g., random-access memory, read-only memory, flash memory), electronic storage unit 815 (e.g., hard disk), communication interface 820 (e.g., network adapter) for communicating with one or more other systems, and peripheral devices 825, such as cache, other memory, data storage and/or electronic display adapters. The memory 810, storage unit 815, interface 820 and peripheral devices 825 are in communication with the CPU 805 through a communication bus (solid lines), such as a motherboard. The storage unit 815 can be a data storage unit (or data repository) for storing data. The computer system 801 can be operatively coupled to a computer network (“network”) 830 with the aid of the communication interface 820. The network 830 can be the Internet, an internet and/or extranet, or an intranet and/or extranet that is in communication with the Internet. The network 830 in some cases is a telecommunication and/or data network. The network 830 can include one or more computer servers, which can enable distributed computing, such as cloud computing. The network 830, in some cases with the aid of the computer system 801, can implement a peer-to-peer network, which may enable devices coupled to the computer system 801 to behave as a client or a server.

The CPU 805 can execute a sequence of machine-readable instructions, which can be embodied in a program or software. The instructions may be stored in a memory location, such as the memory 810. The instructions can be directed to the CPU 805, which can subsequently program or otherwise configure the CPU 805 to implement methods of the present disclosure. Examples of operations performed by the CPU 805 can include fetch, decode, execute, and writeback.

The CPU 805 can be part of a circuit, such as an integrated circuit. One or more other components of the system 801 can be included in the circuit. In some cases, the circuit is an application specific integrated circuit (ASIC).

The storage unit 815 can store files, such as drivers, libraries and saved programs. The storage unit 815 can store user data, e.g., user preferences and user programs. The computer system 801 in some cases can include one or more additional data storage units that are external to the computer system 801, such as located on a remote server that is in communication with the computer system 801 through an intranet or the Internet.

The computer system 801 can communicate with one or more remote computer systems through the network 830. For instance, the computer system 801 can communicate with a remote computer system of a user (e.g., a mobile computing device). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 801 via the network 830.

Methods as described herein can be implemented by way of machine (e.g., computer processor) executable code stored on an electronic storage location of the computer system 801, such as, for example, on the memory 810 or electronic storage unit 815. The machine executable or machine readable code can be provided in the form of software. During use, the code can be executed by the processor 805. In some cases, the code can be retrieved from the storage unit 815 and stored on the memory 810 for ready access by the processor 805. In some situations, the electronic storage unit 815 can be precluded, and machine-executable instructions are stored on memory 810.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 801, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 801 can include or be in communication with an electronic display 835 that comprises a user interface (UI) 840 for providing, for example, an interface for modifying machine learning parameters. Examples of UI' s include, without limitation, a graphical user interface (GUI) and web-based user interface.

Methods and systems of the present disclosure can be implemented by way of one or more algorithms. An algorithm can be implemented by way of software upon execution by the central processing unit 805. The algorithm can, for example, determine a POI %.

While preferred embodiments of the present disclosures have been shown and described herein, it will be obvious to those skilled in the art that such embodiments are provided by way of example. It is not intended that the present disclosures be limited by the specific examples provided within the specification. While the present disclosures have been described with reference to the aforementioned specification, the descriptions and illustrations of the embodiments herein are not meant to be construed in a limiting sense. Numerous variations, changes, and substitutions will now occur to those skilled in the art without departing from the present disclosures. Furthermore, it shall be understood that all aspects of the present disclosures are not limited to the specific depictions, configurations or relative proportions set forth herein which depend upon a variety of conditions and variables. It should be understood that various alternatives to the embodiments described herein may be employed. It is therefore contemplated that the present disclosures shall also cover any such alternatives, modifications, variations or equivalents. It is intended that the following claims exemplify the scope of the present disclosures and that methods and structures within the scope of these claims and their equivalents be covered thereby.

### Proteins of Interest

Concentrations for any protein of interest (POI) can be estimated by the herein-disclosed methods and systems.

The systems and methods may be used in the context of POI can be naturally expressed by cells of any animal species, plant species, or microbial species, e.g., a fungal species or a bacterial species. In some cases, the POI is a protein naturally expressed by the host cell.

The systems and methods may be used in the context of POI expressed by a cultured host cell, e.g., a plant cell, an animal cell, or a microbial cell (for example a fungal cell or a bacterial cell). In some cases, the host cell is engineered to express the POI, i.e., a recombinant protein.

In some cases, the POI is an enzyme, such as used in processing and/or production of food and/or beverage ingredients and products. Some examples of animal-derived enzymes including trypsin, chymotrypsin, pepsin and pre- and pre-pro-forms of such enzymes (i.e., pepsinogen in the case of pepsin). In some cases, the animal protein is a nutritive protein such as a protein that holds or binds to a vitamin or mineral (e.g., an iron-binding protein or heme binding protein), or a protein that provides a source of protein and/or particular amino acids.

In some cases, the POI may be an egg white protein having a sequence (or variant thereof) derived from a bird selected from the group consisting of poultry, fowl, waterfowl, game bird, chicken, quail, turkey, duck, ostrich, goose, gull, guineafowl, hummingbird, pheasant, emu, and any combination thereof. Illustrative egg white proteins include ovalbumin, ovotransferrin, ovomucoid, ovoglobulin G2, ovoglobulin G3, lysozyme, ovoinhibitor, ovoglycoprotein, flavoprotein, ovomacroglobulin, ovostatin, cystatin, avidin, ovalbumin related protein X, and ovalbumin related protein Y. As examples, the ovalbumin may have the sequence of a chicken ovalbumin and the lysozyme may have the sequence of a goose lysozyme. In some embodiments, the POI is a variant of the egg white protein, e.g., having a sequence identity of at least 80%, 90%, 95%, 96%, 97%, 98%, 99% or 99.5% to the natural protein.

In some embodiments, the POI is a protein that naturally occurs in a hen egg white; for example, ovalbumin, ovotransferrin, ovomucoid, ovoglobulin G2, ovoglobulin G3, lysozyme, ovoinhibitor, ovoglycoprotein, flavoprotein, ovomacroglobulin, ovostatin, cystatin, avidin, ovalbumin related protein X, and ovalbumin related protein Y. In some embodiments, the POI is a variant of a protein that naturally occurs in a hen egg white, e.g., having a sequence identity of at least 80%, 90%, 95%, 96%, 97%, 98%, 99% or 99.5% to the natural protein.

In some cases, a host cell expresses a plurality of POIs.

### AAA and Autopanning Experiment

The following paragraphs describe an experimental example and should not be construed to limit any aspect of the preceding disclosure.

### Abstract

Amino acid analysis (AAA) fingerprint modeling for % POI estimation can accelerate autopanning by facilitating quantification and reducing analytical chemistry labor required for new assay development prior to officially starting a new protein program.

### Introduction and Prior Work

Autopanning refers to in-silico screening modeling which predicts if a new protein will see high expression. A method like high-performance liquid chromatography (HPLC) could be used to determine a protein concentration (“titer”) to evaluate if the POI is high or low expressing. But developing a new HPLC assay and selecting standards may take about two months. As autopanning may use expert review of gel images to determine high or low expression, evaluation of a protein may rely on expert, but subjective, evaluation given the infeasibility of developing hundreds of these new assays.

A machine learning method may be used for estimating what percentage of a protein sample is a POI without requiring experimental data on that new POI. The method may use an amino acid analysis (AAA) fingerprint with a synthetic simulated dataset.

### Method

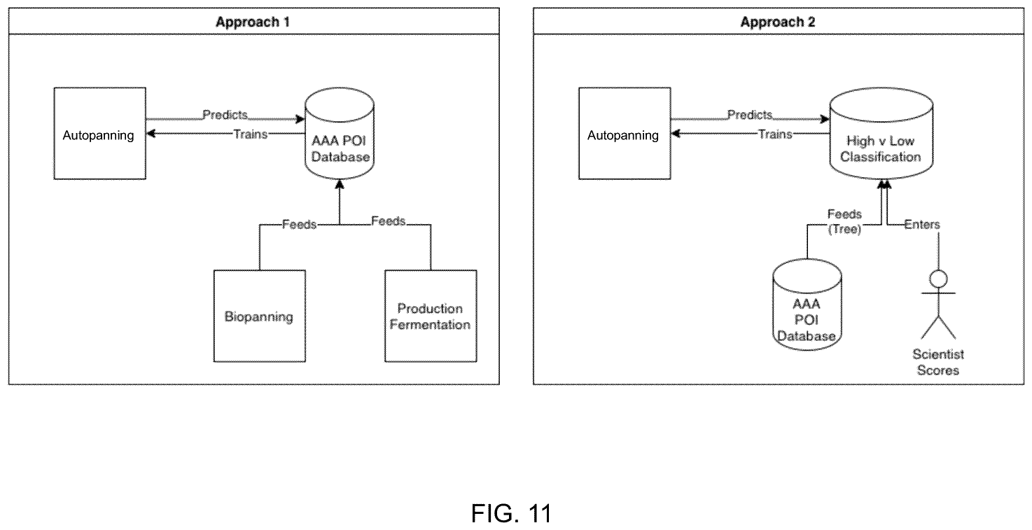

FIG. 11 illustrates two approaches for using AAA with a synthetic simulated dataset. Instead of using gel image scores from 0 to 3, autopanning can retrain on high vs low expression based on AAA predictions (“Approach 1”). This may require a large diversity of samples but could offer a more automated signal, saving internal scientists tens of hours reviewing gel images. Alternatively, autopanning can continue to use the 0 to 3 scores (which consider the “sharpness” of the POI bands) can use a mixture of human generated gel image scores and AAA results (“Approach 2”). This can be done via a decision tree model stratifying based on a single input: AAA-predicted POI concentration.

To demonstrate the viability of this second approach, the disclosed experiment briefly considers the historical AAA samples for 3 different POIs to show that a relationship exists between the model's predictions and the gel image scores.

### Results

This experiment evaluates the feasibility of its method by examining gel score in relation to previous AAA results. Chicken OVA (ggOVA) sees a gel score of 2 while chicken OVD (ggOVD) sees a gel score of 3. For each, the seven most recent AAA upstream results are reported for those POIs.

| | Gel Score | Mean POI Titer | Median POI Titer |
|---|---|---|---|
| POI | (0-3) | (mg/mL via AAA) | (mg/mL via AAA) |
| ggOVA | 2 | 8.0 | 8.1 |
| ggOVD | 3 | 20.1 | 20.1 |

A Mann-Whitney U test confirms that OVA sees lower concentrations than OVD when using AAA (p<0.05).

### Discussion and Conclusion

This experiment demonstrates the viability of using AAA to both reduce scientist labor in generating gel image scores and may offer “objective” and “quantified” approach to determining if a new POI is high or low expressing. Future work may consider either blending scientist scores with AAA results via Approach 2 or moving over to the AAA database directly via Approach 1. Future work may evaluate this for other POIs. Finally, this experiment suggests that, with sufficient data on FTIR (Fourier Transform Infrared) for POI titer quantification, that this other input data type would work in a similar way to AAA.

### DEFINITIONS

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art. The terminology used herein is for the purpose of describing particular cases only and is not intended to be limiting.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used herein, the singular forms “a”, “an,” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.” As such, this statement is intended to serve as antecedent basis for use of such exclusive terminology as “solely,” “only,” and the like in connection with the recitation of claim elements or use of a “negative” limitation.

As used herein, the term “comprise” or variations thereof such as “comprises” or “comprising” are to be read to indicate the inclusion of any recited feature but not the exclusion of any other features. Thus, as used herein, the term “comprising” is inclusive and does not exclude additional, unrecited features. In some embodiments of any of the compositions and methods provided herein, “comprising” may be replaced with “consisting essentially of” or “consisting of.” The phrase “consisting essentially of” is used herein to require the specified feature(s) as well as those which do not materially affect the character or function of the claimed disclosure. As used herein, the term “consisting” is used to indicate the presence of the recited feature alone.

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the given value. In another example, “about” can mean 10% greater than or less than the stated value. Where particular values are described in the application and claims, unless otherwise stated the term “about” should be assumed to mean an acceptable error range for the particular value. In some instances, the term “about” also includes the particular value. For example, “about 5” includes 5.

The term “substantially” is meant to be a significant extent, for the most part; or essentially. In other words, the term substantially may mean nearly exact to the desired attribute or slightly different from the exact attribute. Substantially may be indistinguishable from the desired attribute. Substantially may be distinguishable from the desired attribute but the difference is unimportant or negligible.

The term “sequence identity” as used herein in the context of amino acid sequences is defined as the percentage of amino acid residues in a candidate sequence that are identical with the amino acid residues in a selected sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, ALIGN-2 or Megalign (DNASTAR) software. Those skilled in the art can determine appropriate parameters for measuring alignment, including any algorithms needed to achieve maximal alignment over the full-length of the sequences being compared.

Any aspect or embodiment described herein can be combined with any other aspect or embodiment as disclosed herein.

1. A computer-implemented method for estimating protein concentrations in one or more heterogeneous samples, comprising:
generating a synthetic dataset based at least on protein signature or fingerprint data;
training a model using in part the synthetic dataset, without requiring protein-specific calibration or training; and
using the model to estimate or predict a percentage amount of a specific protein of interest (POI) in one or more heterogeneous samples.

2. The method of claim 1, wherein the protein signature or fingerprint data comprises amino acid analysis (AAA) data.

3. The method of claim 1, wherein the protein signature or fingerprint data comprises high performance liquid chromatography (HPLC) or infrared spectroscopy (IR)-based data.

4. The method of claim 1, wherein the model is useable to predict or estimate a plurality of different POIs in a plurality of different heterogeneous samples.

5. The method of claim 1, further comprising using the model to predict one or more POIs that are not present in the synthetic dataset or that are not used in training the model.

6. The method of claim 1, wherein the POI % is estimated or predicted using the model in substantially less time, or in a day or less, and utilizing substantially less resources compared to high performance liquid chromatography (HPLC).

7. The method of claim 2, wherein the POI % is estimated or predicted by the model using amino acid, mass, or a mole percentage (%) of the specific POI.

8. The method of claim 2, wherein the AAA data comprises amino acid mass, or an amino acid mole percentage (%) distributions.

9. The method of claim 8, wherein the amino acid mole % distributions are obtained from a set of FASTA files.

10. The method of claim 2, wherein the AAA data comprises theoretical AAA results at 100% purity.

11. The method of claim 2, wherein the synthetic dataset is generated through simulations by combining theoretical AAA values of different proteins expected at 100% purity, at different concentrations of the different proteins.

12. The method of claim 2, wherein the synthetic dataset comprises weighted averages of theoretical AAA values of different proteins at 100% purity, wherein the weighted averages are generated by randomly applying a plurality of weights to the theoretical AAA values.

13. The method of claim 2, wherein the synthetic dataset comprises more than 1000 simulated theoretical AAA results.

14. The method of claim 2, wherein the training of the model is performed within one hour or less.

15. The method of claim 2, wherein the model comprises a neural network.

16. The method of claim 15, wherein the neural network is based in part on a pseudo-Siamese architecture.

17. The method of claim 16, wherein the pseudo-Siamese neural network architecture comprises a pair of input vectors without corresponding parallel branches.

18. The method of claim 17, wherein the pair of input vectors comprises (i) a first input vector comprising of historical or available AAA results and (ii) a second input vector comprising theoretical AAA results at 100% purity.

19. The method of claim 18, wherein the historical or available AAA results are obtained from a first database of naturally occurring proteins inside hen egg white and a second database of common host cell proteins.

20. The method of claim 19, wherein the host cell proteins are expressed by a group of microbes selected from a Pichia species, a Saccharomyces species, a Trichoderma species, a Pseudomonas species, an Aspergillus species, and an E. coli species.

21. The method of claim 20, wherein the Pichia species is Pichia pastoris or the Saccharomyces species is Saccharomyces cerevisiae.

22. The method of claim 15, wherein the neural network does not require learning of a lower dimensional representation.

23. The method of claim 15, wherein a comparison function in the neural network is automatically learned without external human input or intervention.

24. The method of claim 15, wherein generating the synthetic dataset further comprises splitting the synthetic dataset into a training set, a validation set, and a test set.

25. The method of claim 24, wherein training the model comprises using the training set in fitting the model.

26. The method of claim 24, wherein the test set is not provided to the model during the training of the model.

27. The method of claim 24, further comprising: using the validation set to check a mean squared error (MAE) of the model and determining whether the MAE of the model meets a criteria threshold.

28. The method of claim 27, further comprising: persisting the model to memory upon determining that the MAE of the model meets the criteria threshold.

29. The method of claim 1, wherein the model has a performance of a mean absolute error (MAE) of 3 points for a hidden test set of proteins within the synthetic dataset, and 6 points for novel proteins that are not present in the synthetic dataset.

30. The method of claim 1, wherein the model is based on linear (lasso), support vector machine (SVM), decision tree, or random forest.

31. The method of claim 1, wherein the POI % is greater than or equal to about 50%.

32. The method of claim 1, wherein the POI % is less than about 50%.

33. The method of claim 1, wherein the model is further trained using actual or real data collected over time.

34. The method of claim 1, wherein the specific POI is a target product.

35. The method of claim 34, wherein the target product is a protein recombinantly expressed by a host cell.

36. The method of claim 33, wherein the specific POI is a contaminant.

37. The method of claim 36, wherein the contaminant is unintentionally included in the multi-protein sample.

38. The method of claim 33, wherein the specific POI is a process byproduct or an added protein.

39. A system for estimating protein concentrations in one or more heterogeneous samples, the system comprising:
one or more processors; and
a non-transitory computer readable medium storing a plurality of instructions, which when executed, causes the one or more processors to:
(a) generate a synthetic dataset based at least on protein signature or fingerprint data;
(b) perform training of a model using the synthetic dataset, without requiring protein-specific calibration or training; and
(c) estimate or predict, using the model, a percentage amount of a specific protein of interest (POI) in the one or more heterogeneous samples.

40. The system of claim 39, wherein the specific POI is a target product.

41. The system of claim 39, wherein the target product is a protein recombinantly expressed by a host cell.

42. The system of, claim 39 wherein the specific POI is a contaminant.

43. The system of claim 42, wherein the contaminant is unintentionally included in the multi-protein sample.

44. The system of claim 39, wherein the specific POI is a process byproduct or an added protein.

45. The system of claim 39, wherein the multi-protein sample comprises a culturing medium for cultivating a host cell or the multi-protein sample is derived from a culturing medium used for cultivating a host cell.

46. The system of claim 45, wherein the host cell is a microbial cell selected from a Pichia cell, a Saccharomyces cell, a Trichoderma cell, a Pseudomonas cell, an Aspergillus cell, and an E. coli cell.

47. The system of claim 46, wherein the Pichia cell is a Pichia pastoris cell or the Saccharomyces cell is a Saccharomyces cerevisiae cell.

48. A computer-implemented method for estimating protein concentrations in one or more heterogeneous samples, comprising:
using a model to estimate or predict a percentage amount of a specific protein of interest (POI) in one or more heterogeneous samples, wherein the model is obtained by:
(a) generating a synthetic dataset based at least on protein signature or fingerprint data; and
(b) training the model using in part the synthetic dataset, without requiring protein-specific calibration or training.

49. The method of claim 48, wherein the protein signature or fingerprint data comprises amino acid analysis (AAA) data.

50. The method of claim 48, wherein the protein signature or fingerprint data comprises high performance liquid chromatography (HPLC) or infrared spectroscopy (IR)-based data.

51. The method of claim 48, wherein the model is useable to predict or estimate a plurality of different POIs in a plurality of different heterogeneous samples.

52. The method of claim 48, further comprising using the model to predict one or more POIs that are not present in the synthetic dataset or that are not used in training the model.

53. The method of claim 48, wherein the POI % is estimated or predicted using the model in substantially less time, or in a day or less, and utilizing substantially less resources compared to high performance liquid chromatography (HPLC), which normally takes about a month to establish calibration for each per protein examined.

54. The method of claim 49, wherein the POI % is estimated or predicted by the model using amino acid, mass, or a mole percentage (%) of the specific POI.

55. The method of claim 49, wherein the AAA data comprises amino acid, mass, or a mole percentage (%) distributions.

56. The method of claim 55, wherein the amino acid mole % distributions are obtained from a set of FASTA files.

57. The method of claim 49, wherein the AAA data comprises theoretical AAA results at 100% purity.

58. The method of claim 49, wherein the synthetic dataset is generated through simulations by combining theoretical AAA values of different proteins expected at 100% purity, at different concentrations of the different proteins.

59. The method of claim 49, wherein the synthetic dataset comprises weighted averages of theoretical AAA values of different proteins at 100% purity, wherein the weighted averages are generated by randomly applying a plurality of weights to the theoretical AAA values.

60. The method of claim 49, wherein the synthetic dataset comprises more than 1000 simulated theoretical AAA results.

61. The method of claim 49, wherein the training of the model is performed in one hour or less.

62. The method of claim 49, wherein the model comprises a neural network.

63. The method of claim 62, wherein the neural network is based in part on a pseudo-Siamese architecture.

64. The method of claim 63, wherein the pseudo-Siamese neural network architecture comprises a pair of concatenated input vectors without corresponding parallel branches.

65. The method of claim 64, wherein the pair of concatenated input vectors comprises (i) a first input vector comprising of historical or available AAA results and (ii) a second input vector comprising theoretical AAA results at 100% purity.

66. The method of claim 65, wherein the historical or available AAA results are obtained from a first database of naturally occurring proteins inside hen egg white and a second database of common host cell proteins.

67. The method of claim 66, wherein the host cell proteins are expressed by a microbe selected from Pichia species, a Saccharomyces species, a Trichoderma species, a Pseudomonas species, an Aspergillus species, and an E. coli species.

68. The method of claim 67, wherein the Pichia species is Pichia pastoris or the Saccharomyces species is Saccharomyces cerevisiae.

69. The method of claim 62, wherein the neural network does not require learning of a lower dimensional representation.

70. The method of claim 62, wherein a comparison function in the neural network is automatically learned without external human input or intervention.

71. The method of claim 48, wherein generating the synthetic dataset further comprises splitting the synthetic dataset into a training set, a validation set, and a test set.

72. The method of claim 71, wherein training the model comprises using the training set in fitting the model.

73. The method of claim 71, wherein the test set is not provided to the model during the training of the model.

74. The method of claim 71, further comprising: using the validation set to check a mean squared error (MAE) of the model and determining whether the MAE of the model meets a criteria threshold.

75. The method of claim 71, further comprising: persisting the model to memory upon determining that the MAE of the model meets the criteria threshold.

76. The method of claim 48, wherein the model has a performance of a mean absolute error (MAE) of 3 points for a hidden test set of proteins within the synthetic dataset, and 6 points for novel proteins that are not present in the synthetic dataset.

77. The method of claim 48, wherein the model is based on linear (lasso), support vector machine (SVM), decision tree, or random forest.

78. The method of claim 48, wherein the POI % is greater than or equal to about 50%.

79. The method of claim 48, wherein the POI % is less than about 50%.

80. The method of claim 48, wherein the model is further trained using actual or real data collected over time.

81. The method of claim 48, wherein the specific POI is a target product.

82. The method of claim 80, wherein the target product is a protein recombinantly expressed by a host cell.

83. The method of claim 80, wherein the specific POI is a contaminant.

84. The method of claim 83, wherein the contaminant is unintentionally included in the multi-protein sample.

85. The method of claim 80, wherein the specific POI is a process byproduct or an added protein.

86. The method of claim 48, wherein the model includes four layers of neurons, wherein the layers are of sizes 64, 32, 16, and 8 neurons.

87. The method of claim 48, wherein the model is trained using a ridge (L2) regularization of 0.1 and an Adam learning rate of 00001.
